(19) United States
(12) Patent Application Publication
(10) Pub. No.: US 2017/0115200 A1
(43) Pub. Date: Apr. 27, 2017

(54) IMAGING A POROUS ROCK SAMPLE USING A NANOPARTICLE SUSPENSION

(71) Applicant: Halliburton Energy Services, Inc., Houston, TX (US)

(72) Inventors: Cheng Chen, Cypress, TX (US); Vladimir Nikolayevich Martysevich, Spring, TX (US); Brady Kevin Crane, Atascocita, TX (US); Scott Thomas Squires, Spring, TX (US)

(73) Assignee: Halliburton Energy Services, Inc., Houston, TX (US)

(21) Appl. No.: 15/317,091

(22) PCT Filed: Jul. 11, 2014
(86) PCT No.: PCT/US14/46342

§ 371 Date: Dec. 7, 2016

(51) Int. Cl. G01N 15/08; G01N 33/24; G06T 7/60; G01N 23/04

(52) U.S. Cl. CPC G01N 15/082; G01N 15/088; G01N 23/046; G01N 33/24; G06T 7/60; G01N 2015/0846; G01N 2223/616; G01N 2223/401; G06T 2207/10081; G06T 2207/30181

(57) ABSTRACT

Some aspects of what is described here relate to analyzing a porous rock sample. A suspension of nanoparticles is injected into a rock sample. The rock sample includes pore space defined within a rock medium. An image of the nanoparticles in the pore space is obtained, for example, by three-dimensional x-ray tomography or another non-destructive imaging technique. The pore space of the rock sample is analyzed based on the image. For example, the porosity and permeability of the rock sample can be computed in some cases.

### BACKGROUND

The following description relates to imaging a porous rock sample using a nanoparticle suspension.

Rock samples are often extracted from subterranean rock formations and analyzed in a laboratory setting to gain information about the properties of the subterranean rock formation. For example, a core sample can be extracted from a well bore defined in the subterranean region or from an outcropping or another location. In some instances, rock samples are analyzed to provide an estimate of porosity, permeability, density, or other properties of the subterranean rock formation.

DESCRIPTION OF DRAWINGS

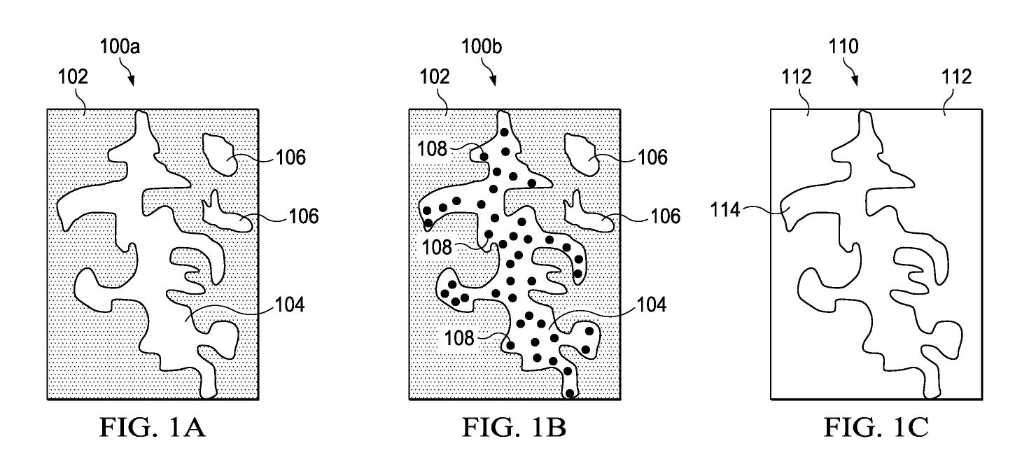

FIG. 1A is a schematic diagram of an example rock sample.

FIG. 1B is a schematic diagram of the example rock sample of FIG. 1A with an injected nanoparticle suspension.

FIG. 1C is an example image of the rock sample of FIG. 1B.

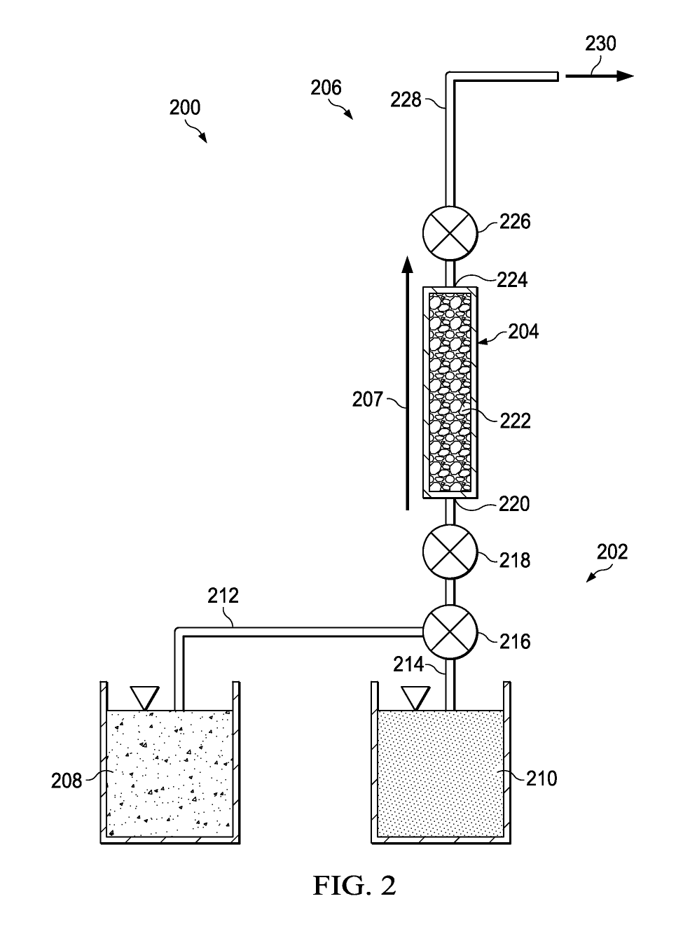

FIG. 2 is a schematic diagram of an example sample preparation system.

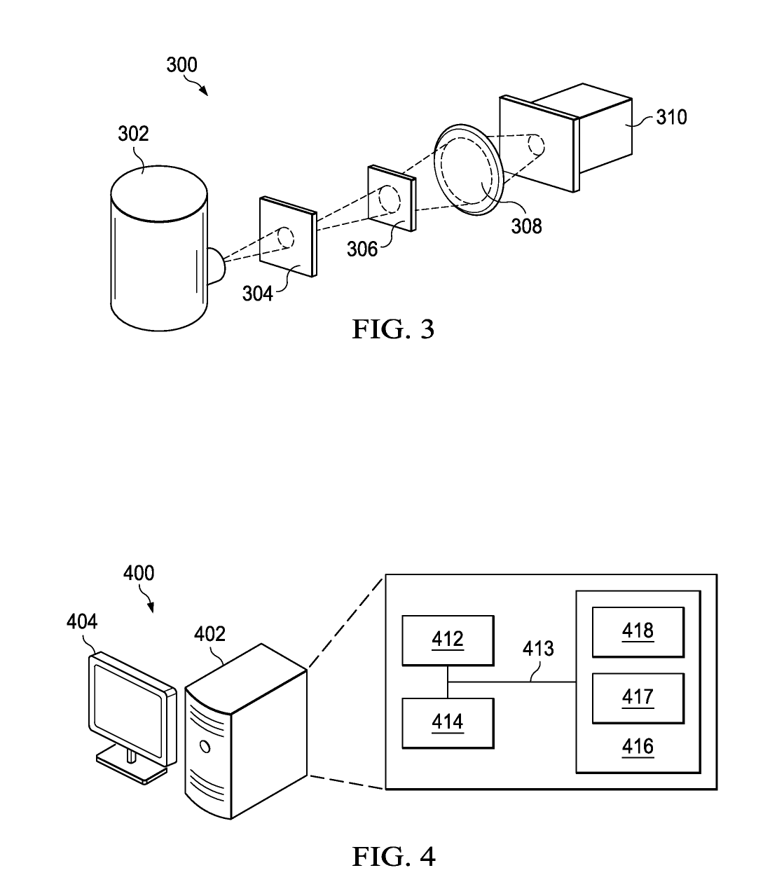

FIG. 3 is a schematic diagram of an example imaging system.

FIG. 4 is a schematic diagram of an example data analysis system.

Like reference symbols in the various drawings indicate like elements.

### DETAILED DESCRIPTION

In some implementations of what is described here, porosity and permeability of porous media are measured using nanoparticles and x-ray tomography. In some instances, other properties of the porous media can be measured, and other types of imaging and analysis can be performed. The porous media can be, for example, shale, sandstone, coal, or another type of rock extracted from a subterranean region. In some cases, the techniques described here can improve porosity and permeability measurement for tight reservoirs and other types of formations.

The nanoparticles can serve as an x-ray contrast tracer to mark the effective porosity, i.e., porosity that provides permeability for fluid flow. In some cases, the nanoparticles are dispersed in an aqueous phase, and the surface tension and capillary forces are attenuated or negligible. The nanoparticles can enter nanometer scale pores and throats easily, giving rise to accurate measurements of nanometer-scale porosity. The measurements can isolate the effective porosity (i.e., the connected porous space) from the bulk porosity (which includes the isolated pore space). And a more accurate computation of pore geometry can allow subsequent calculations of permeability, pore and throat size distributions, connectivity, and tortuosity, and other parameters. Measurement can be done on rock cuttings with irregular size and geometry. Therefore, the techniques described here can be used for a wide range of rock samples, including cores samples with poor cutting quality.

In some aspects, a sample preparation system injects a suspension of nanoparticles into a sample of porous rock. After the nanoparticles diffuse into the connected pore space defined in the rock medium, an imaging system (e.g., x-ray tomography system, magnetic resonance imaging system, etc.) obtains an image of the nanoparticles in the pore space. The nanoparticles act as an imaging-contrast agent, and the porous regions within the rock sample that are occupied by the nanoparticles can be identified from the image. The pore space is then analyzed based on the image. For example, tomography can be used to extract quantitative information. In some cases, the image data are used to calculate porosity and permeability of the rock sample. In some cases, the image data are used to identify connectivity among pore spaces in the rock sample, or to identify the spatial distribution of connected pore spaces in the rock sample.

In some implementations, the sample preparation system is a fluid injection system that includes a column or another housing for the rock sample. A flow inlet through the housing communicates the suspension of nanoparticles into the pore space in the rock medium, and a flow outlet through the housing communicates the suspension of nanoparticles from the rock sample. An example of a sample preparation system is shown in FIG. 2.

In some implementations, the imaging system generates a two-dimensional or three-dimensional image. For example, three-dimensional x-ray tomography can be used. An example of an x-ray tomography system is shown in FIG. 3. The imaging system generates the image in a non-destructive manner, i.e., without destructing the rock sample.

In some implementations, a data analysis system analyzes the image of the rock sample. The data analysis system can include a computer system, for example, that can run image analysis software, flow simulation software, or other types of software. In some cases, the rock medium has a first mass density, and the nanoparticles have a higher mass density, and the images are analyzed based on mass density contrast in the image. In some cases, the permeability is computed based on a flow simulation of the connected pore space geometry extracted from the image.

FIGS. 1A and 1B are schematic diagrams of an example rock sample. The rock sample 100a in FIG. 1A includes pore spaces defined in a rock medium 102. The pore spaces in the rock medium 102 include connected pore space 104 and isolated pore spaces 106. The isolated pore spaces 106 do not directly communicate flow with the connected pore space 104. In some instances, fluids can invade the isolated pore spaces 106 from the rock medium 102 surrounding the isolated pore spaces 106.

The example rock sample 100b in FIG. 1B is the rock sample 100a of FIG. 1A with an injected nanoparticle suspension. The nanoparticle suspension includes a mixture of nanoparticles 108 and a carrier fluid (e.g., liquid, gas, or a combination). In some examples, the nanoparticle suspension is injected into the porous rock medium through pores exposed on an outer surface of the rock sample. The carrier fluid and the nanoparticles 108 flow from the exposed pores into the connected pore space 104. In the example shown in FIG. 1B, the nanoparticles migrate into and occupy all the connected pore space 104 by Brownian motion (i.e., by a diffusion-based process). Although the carrier fluid may, in some cases, be capable of permeating the rock medium 102 and entering the isolated pore spaces 106 through the rock medium 102, the nanoparticles 108 are too large to permeate the rock medium 102. Therefore, the nanoparticles 108 only invade the connected pore space 104 without entering the isolated pore spaces 106.

The nanoparticles 108 can act as an imaging contrast agent in the rock sample 100b. As such, the nanoparticles 108 and their properties can be selected based on an imaging process that will be used to obtain an image of the rock sample. For example, the nanoparticles 108 can be designed to have a mass-density that is substantially higher than the rock medium and the carrier fluid, so that the nanoparticles 108 are distinguishable in an x-ray image of the rock sample 100b. As another example, the nanoparticles 108 can be designed to have a nuclear-spin isotope concentration that is substantially higher than the rock medium and the carrier fluid, so that the nanoparticles 108 are distinguishable in a magnetic resonance imaging (MRI) image of the rock sample 100b.

In some examples, the nanoparticles 108 have a light-weight or hollow core and a high-density outer shell. The high-density outer shell can be, for example, a non-reactive metal material (e.g., gold, silver, etc.) that provides contrast in mass-density based imaging platforms. The light-weight core can be, for example, carbon or a carbon-based material. The light-weight or hollow core reduces the mass of the nanoparticles, which increases their flow rate within the connected pore space 104. In some cases, the nanoparticles are solid and have a relatively uniform density over their volume. The material and size of the nanoparticles 108 can be selected based on the imaging platform that will be used, the type of rock sample, the average pore size in the rock sample, the type of carrier fluid, or a combination of these and other factors. For example, larger nanoparticles 108 can be used to study larger pore space of a sand stone rock sample. In some examples, the nanoparticles 108 can have a diameter in the range of 5 to 500 nanometers, or nanoparticles of another size can be used. In some cases, the rock sample is part of a core sample from a subterranean region that contains hydrocarbon fluid (e.g., oil, natural gas, etc.), and the nanoparticles 108 are selected based on the type of hydrocarbon fluid. For example, smaller nanoparticles can be used to study rock formations that contain lower viscosity natural gas, and larger nanoparticles can be used to study rock formations that contain higher viscosity oil.

The carrier fluid in the nanoparticle suspension can include gas, liquid, or a combination of multiple fluid types. In some cases, the carrier fluid includes nitrogen gas, helium gas, air, natural gas, water, or a combination of these or other fluids. In some cases, the carrier fluid has a low viscosity or other properties that promote flow into small pore spaces in the rock sample.

FIG. 1C is an example image 110 of the rock sample 100b of FIG. 1B. The image 110 can be generated in a non-destructive, non-invasive manner, for example, by a three-dimensional x-ray tomography scanner. The nanoparticles 108 have a density that is different from (e.g., higher or lower than) the density of the rock medium 102 and the carrier fluid. Therefore the nanoparticles 108 act as an imaging contrast agent in a density-based image of the rock sample 100b. In other words, the nanoparticles 108 are distinguishable from the rock medium 102 and the carrier fluid in the image 110 of the rock sample 100b.

In the example shown in FIG. 1C, the image 110 includes a first region 112 and a second region 114. The second region 114 corresponds to the connected pore space 104 where the nanoparticles 108 have invaded the rock sample 100b, and the first region 112 corresponds to the other portions (the isolated pore spaces 106 and the rock medium 102) of the rock sample 100b, which were not invaded by a significant number of nanoparticles 108. Accordingly, the image 110 can be analyzed (e.g., by image analysis software or otherwise) to identify the connected pore space 104 in the rock sample 100b. For example, the effective porosity can be extracted by three-dimensional reconstruction.

In some example implementations, nanoparticles 108 that have a different mass-density from the rock medium 102 are selected. A suspension of the nanoparticles is injected into the rock sample (e.g., using the sample preparation system 200 shown in FIG. 2 or another type of system), and the nanoparticles are allowed time to migrate into the pores and throats of the connected pore space 104. The wait time is based on the diffusion rate of the nanoparticles, which is generally related to the size of the nanoparticles and the density of the fluid. The rock sample 100b can then be sealed on all sides and scanned (e.g., using the imaging system 300 shown in FIG. 3 or another type of system). Because the nanoparticles have different density from the rock medium 102, the pore space occupied by the particles can be visualized and extracted by the imaging system. The effective porosity can be computed based on the image 110, for example, by normalizing the nanoparticle-occupied pore volume (represented by the second region 114) by the total sample volume (represented by the first region 112). Permeability can be computed, for example, by lattice Boltzmann simulation using the connected pore geometry.

FIG. 2 is a schematic diagram of an example sample preparation system 200. At a high level, the sample preparation system 200 includes a fluid inflow system 202, a sample housing system 204, and a fluid outflow system 206. The arrow 207 shows the general direction of flow in the sample preparation system 200. The sample housing system 204 can include a cylindrical column or another type of housing made of plastic or another light-weight material. In some cases, the rock sample 222 is held in the same housing during a subsequent imaging process; and the low density of the housing wall provides low x-ray attenuation, which is favorable for a high signal-to-noise ratio in some imaging processes (e.g., in a CT scan). A sample preparation system may include additional or different features, and the features of a sample preparation system can be arranged as shown in FIG. 2 or in another manner.

In the example shown, the fluid inflow system 202 includes a nanoparticle suspension 208 in a first fluid reservoir, a background solution 210 in a second reservoir, and a flow system connecting the first and second reservoirs to the sample housing system 204. The flow system includes two flow channels 212, 214 connected to two respective inlets of a three-way valve 216. The outlet of the three-way valve is connected to an inlet of a two-way valve 218; an outlet of the two-way valve is connected to the sample housing system 204. The three-way valve 216 can receive a flow of the nanoparticle suspension 208 from the first reservoir, a flow of the background solution 210 from the second reservoir, or possibly both. The three-way valve 216 communicates flow to the two-way valve 218, which communicates flow to the sample housing system 204.

In the example shown, the sample housing system 204 houses a rock sample 222. For example, the rock sample 222 can be the rock sample 100b shown in FIG. 1B or another type of rock sample. The sample housing system 204 includes an inlet 220 that communicates flow from the two-way valve 218 into pore spaces in the rock sample 222. The sample housing system 204 includes an outlet 224 that communicates flow from the rock sample 222 to the fluid outflow system 206. The fluid outflow system 206 includes a two-way valve 226, a flow channel 228, and an outflow of waste fluid 230. The two-way valve 226 receives fluid from the outlet 224 of the sample housing system 204 and communicates flow through the flow channel 228 to a waste reservoir or another external system.

In an example aspect of operation, the rock sample 222 is machined and polished, and then placed in a column of the sample housing system 204. The two-way valves 218, 226 are then connected to the top and bottom of the column, and the column is placed in the flushing system as shown in FIG. 2. The three-way valve 216 is switched to the reservoir of the background solution 210. The background solution can be, for example, pure water or another solution containing stabilization agents, to mitigate nanoparticle aggregation. Typically, after a few pore volumes of flushing driven by a pump, the porous medium in the column has been saturated with the background solution. The three-way valve 216 is then switched to the reservoir of nanoparticle suspension 208, and the pump is used to flush the nanoparticle suspension 208 through the column. A low flow rate can be used, for example, such that diffusion dominates the nanoparticle transport. The flushing can continue for a time duration, for example, so that the nanoparticles diffuse into smaller pores in the porous medium. The pump is then stopped, and the two-way valves 218, 226 at the top and bottom of the column are disconnected. The column (with valves connected to prevent water evaporation) is then transported to the imaging system (e.g., to a CT scanner for an X-ray tomography scan).

FIG. 3 is a schematic diagram of an example imaging system 300. The example imaging system 300 shown in FIG. 3 is an example of an x-ray tomography system. The example x-ray tomography system includes an x-ray source 302, an imaging sample 304 (e.g., the column containing the rock sample 222 from FIG. 2, or another type of imaging sample), a scintillator screen 306, high-resolution detector optics 308, and a CCD camera 310. An x-ray tomography system can include additional or different features, and the features of an x-ray tomography system can operate as shown or in another manner.

In the example shown in FIG. 3, the x-ray source 302 produces x-rays that are directed to the imaging sample 304. The x-rays interact with the imaging sample 304 and strike the scintillator screen 306. The scintillator screen 306 produces an optical output based on the x-rays from the imaging sample 304. The high-resolution detector optics 308 optically detect the output from the scintillator screen 306. The CCD camera 310 records the optical information captured by the high-resolution detector optics components 308. The CCD camera 310 produces an image or series of images based on the recorded information. The images can be two-dimensional, three-dimensional, etc. The image obtained by the imaging system 300 can be provided to a data analysis system for processing.

FIG. 4 is a schematic diagram of an example data analysis system 400 that includes a computer system 402 and a display device 404. The data analysis system 400 can be located at a data-processing center, a computing facility, or another location. The example data analysis system 400 can communicate with (e.g., send data to or receive data from) an imaging system. For example, the data analysis system 400 may receive image data from the imaging system 300 in FIG. 3 or another type of imaging system. In some examples, all or part of the data analysis system 400 may be included with or embedded in an imaging system. The data analysis system 400 or any of its components can be located with or apart from an imaging system.

In some implementations, the data analysis system 400 can include or be implemented on various types of devices, including, but not limited to, personal computer systems, desktop computer systems, laptops, mainframe computer systems, handheld computer systems, application servers, computer clusters, distributed computing systems, workstations, notebooks, tablets, storage devices, or another type of computing system or device.

In some examples, all or part of the data analysis system 400 communicates with an imaging system or another external system over a communication link. The communication links can include wired or wireless communication networks, other types of communication systems, or a combination thereof. For example, the computer system 402 may include or have access to a telephone network, a data network, a satellite system, dedicated hard lines, or other types of communication links.

The example display device 404 can produce a visual output. The display device 404 can include a computer monitor (e.g., LCD screen), a projector, a printer, a touchscreen device, a plotter, or a combination of one or more of these. In some instances, the display device 404 displays images obtained by an imaging system, plots of data obtained by analyzing images, or other types of information.

As shown in the schematic diagram in FIG. 4, the example computer system 402 includes a memory 416, a processor 414, and input/output controllers 412 communicably coupled by a bus 413. A computing system can include additional or different features, and the components can be arranged as shown or in another manner. The memory 416 can include, for example, a random access memory (RAM), a storage device (e.g., a writable read-only memory (ROM) or others), a hard disk, or another type of storage medium. The computer system 402 can be preprogrammed or it can be programmed (and reprogrammed) by loading a program from another source (e.g., from a CD-ROM, from another computer device through a data network, or in another manner).

In some examples, the input/output controllers 412 are coupled to input/output devices (e.g., a monitor, a mouse, a keyboard, or other input/output devices) and to a network. The input/output devices can communicate data in analog or digital form over a serial link, a wireless link (e.g., infrared, radio frequency, or others), a parallel link, or another type of link. The network can include any type of communication channel, connector, data communication network, or other link. For example, the network can include a wireless or a wired network, a Local Area Network (LAN), a Wide Area Network (WAN), a private network, a public network (such as the Internet), a WiFi network, a network that includes a satellite link, or another type of data communication network.

The memory 416 can store instructions (e.g., computer code) associated with an operating system, computer applications, and other resources. The memory 416 can also store application data and data objects that can be interpreted by one or more applications or virtual machines running on the computer system 402. As shown in FIG. 4, the example memory 416 includes data 418 and applications 417. The data 418 can include image data, rock material data, nanoparticle data, or other types of data. The applications 417 can include image analysis software, simulation software, or other types of applications. In some implementations, a memory of a computing device includes additional or different data, applications, models, or other information.

The processor 414 can execute instructions, for example, to generate output data based on data inputs. For example, the processor 414 can run the applications 417 by executing or interpreting the software, scripts, programs, functions, executables, or other modules contained in the applications 417. The input data received by the processor 414 or the output data generated by the processor 414 can include any of the treatment data, the geological data, the fracture data, the seismic data, or other information.

Some of the subject matter and operations described in this specification can be implemented in digital electronic circuitry, or in computer software, firmware, or hardware, including the structures disclosed in this specification and their structural equivalents, or in combinations of one or more of them. Some of the subject matter described in this specification can be implemented as one or more computer programs, i.e., one or more modules of computer program instructions, encoded on a computer storage medium for execution by, or to control the operation of, data-processing apparatus. A computer storage medium can be, or can be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The term “data-processing apparatus” encompasses all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them.

A computer program (also known as a program, software, software application, script, or code) can be written in any form of programming language, including compiled or interpreted languages, declarative or procedural languages. A computer program may, but need not, correspond to a file in a file system. A program can be stored in a portion of a file that holds other programs or data (e.g., one or more scripts stored in a markup language document), in a single file dedicated to the program, or in multiple coordinated files (e.g., files that store one or more modules, sub programs, or portions of code). A computer program can be deployed to be executed on one computer or on multiple computers that are located at one site or distributed across multiple sites and interconnected by a communication network.

Some of the processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and processors of any kind of digital computer. Generally, a processor will receive instructions and data from a read-only memory or a random-access memory or both. A computer can include a processor that performs actions in accordance with instructions, and one or more memory devices that store the instructions and data. A computer may also include, or be operatively coupled to receive data from or transfer data to, or both, one or more mass storage devices for storing data, e.g., magnetic disks, magneto optical disks, or optical disks. However, a computer need not have such devices. Devices suitable for storing computer program instructions and data include all forms of non-volatile memory, media and memory devices, including by way of example semiconductor memory devices (e.g., EPROM, EEPROM, flash memory devices, and others), magnetic disks (e.g., internal hard disks, removable disks, and others), magneto optical disks , and CD ROM and DVD-ROM disks. In some cases, the processor and the memory can be supplemented by, or incorporated in, special purpose logic circuitry.

To provide for interaction with a user, operations can be implemented on a computer having a display device (e.g., a monitor, or another type of display device) for displaying information to the user and a keyboard and a pointing device (e.g., a mouse, a trackball, a tablet, a touch sensitive screen, or another type of pointing device) by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

A computer system may include a single computing device, or multiple computers that operate in proximity or generally remote from each other and typically interact through a communication network. Examples of communication networks include a local area network (“LAN”) and a wide area network (“WAN”), an inter-network (e.g., the Internet), a network comprising a satellite link, and peer-to-peer networks (e.g., ad hoc peer-to-peer networks). A relationship of client and server may arise by virtue of computer programs running on the respective computers and having a client-server relationship to each other.

While this specification contains many details, these should not be construed as limitations on the scope of what may be claimed, but rather as descriptions of features specific to particular examples. Certain features that are described in this specification in the context of separate implementations can also be combined. Conversely, various features that are described in the context of a single implementation can also be implemented in multiple embodiments separately or in any suitable sub-combination.

A number of examples have been described. Various modifications can be made without departing from the scope of the present disclosure. Accordingly, other embodiments are within the scope of the following claims.

What is claimed is:

1. A method of analyzing a porous rock sample, the method comprising:
injecting a suspension of nanoparticles into a rock sample, the rock sample comprising pore space defined in a rock medium;
obtaining an image of the nanoparticles in the pore space of the rock sample; and
analyzing the pore space of the rock sample based on the image.

2. The method of claim 1, wherein obtaining an image comprises performing three-dimensional x-ray tomography of the rock sample.

3. The method of claim 1, comprising waiting for the nanoparticles to diffuse into the pore space before obtaining the image.

4. The method of claim 1, wherein analyzing the pore space comprises calculating porosity of the rock sample.

5. The method of claim 1, wherein analyzing the pore space comprises calculating permeability of the rock sample.

6. The method of claim 1, wherein analyzing the pore space comprises identifying connectivity of the pore space.

7. The method of claim 1, wherein analyzing the pore space comprises identifying a spatial distribution of connected pore space in the rock sample.

8. The method of claim 1, wherein injecting the suspension comprises injecting the suspension through a flow inlet in fluid communication with the pore space.

9. The method of claim 1, wherein obtaining an image comprises non-destructively imaging an interior region of the rock sample.

10. A rock sample analysis system comprising:
an injection system comprising a flow inlet that communicates a suspension of nanoparticles into pore space defined in a rock medium of a rock sample; and
an imaging system that obtains an image of the nanoparticles in the pore space defined in the rock medium.

11. The system of claim 10, further comprising a computing system comprising:
data-processing apparatus; and
memory storing instructions that, when executed by the data-processing apparatus, cause the data-processing apparatus to analyze the pore space based on the image.

12. The system of claim 10, wherein analyzing the pore space comprises at least one of:
calculating porosity of the rock sample;
calculating permeability of the rock sample;
identifying connectivity of the pore space; or
identifying a spatial geometry of connected pore space in the rock sample.

13. The system of claim 10, wherein the imaging system comprises a non-destructive three-dimensional imaging system.

14. The system of claim 10, wherein the injection system further comprises:
a housing that houses the rock sample, wherein the flow inlet comprises an inlet through the housing; and
a flow outlet through the housing that communicates fluid from the rock sample.

15. A method of analyzing pore structure of a rock sample, the method comprising:
receiving an image of a rock sample containing a suspension of nanoparticles injected into the rock sample; and
identifying, based on the image, regions within the rock sample occupied by the nanoparticles; and
analyzing a pore space of the rock sample based on the identified regions.

16. The method of claim 15, wherein the image comprises a three-dimensional image of the rock sample.

17. The method of claim 15, wherein analyzing the pore space comprises calculating at least one of porosity or permeability of the rock sample.

18. The method of claim 15, wherein analyzing the pore space comprises identifying at least one of connectivity of the pore space or geometry of connected pore space in the rock sample.

19. The method of claim 15, wherein the rock sample comprises a rock medium of a first mass density, the nanoparticles comprise material of a second mass density, and the method comprises identifying the regions based on a mass density contrast in the image.
